(19) United States
(12) Patent Application Publication
(10) Pub. No.: US 2019/0183934 A1
(43) Pub. Date: Jun. 20, 2019

(54) TREATMENT OF DISEASE BASED ON IMMUNE CELL SEQUENCING

(71) Applicant: TGM LIFE SCIENCES LLC., Gaithersburg, MD (US)

(72) Inventors: Benjamin M. GREENBERG, Gaithersburg, MD (US); Nancy MONSON, Ovilla, TX (US); Larry TIFFANY, Gaithersburg, MD (US)

(21) Appl. No.: 16/301,073

(22) PCT Filed: May 15, 2017
(86) PCT No.: PCT/US17/32593

§ 371 Date: Nov. 13, 2018

Related U.S. Application Data
(60) Provisional application No. 62/336,017, filed on May 13, 2016.

(51) Int. Cl. A61K 35/17; A61P 25/28; A61K 31/7088; C12N 15/113; A61K 9/51; A61P 35/00; A61K 9/00; A61P 37/00

(52) U.S. Cl. CPC A61K 35/17; A61P 25/28; A61K 31/7088; C12N 15/1138; A61P 37/00; A61P 35/00; A61K 9/0053; A61K 9/0019; A61K 9/51

(57) ABSTRACT

The present invention relates to treatment of disease, including treatment of antibody-mediated autoimmune disorders, as well as treatment of other conditions in which antibody therapeutics can be beneficial for targeting destruction of foreign or malignant cells.

### FIELD OF THE INVENTION

The present invention relates to treatment of disease, including treatment of antibody-mediated autoimmune disorders, as well as treatment of other conditions in which antibody therapeutics can be beneficial for targeting destruction of foreign or malignant cells.

### SUMMARY OF THE INVENTION

In certain aspects, the invention provides methods for treating antibody-mediated autoimmune disease, including demyelinatng diseases, as well as treatment of other conditions in which antibody therapeutics can be beneficial for targeting destruction of foreign or malignant cells.

In various embodiments, the methods involve determining nucleotide sequences in a patient sample for a plurality of immunoglobulin-encoding genes or transcripts, and particularly the nucleotide sequences encoding at least a portion of the complementarity-determining regions (CDRs), which are representative of the antigen-binding specificity. By identifying nucleotide sequences that correspond to the disease, therapeutics can be designed or developed, including oligonucleotide or antibody therapeutics (e.g., to specifically target the patient's pathological antibody component).

For example, in some embodiments, the patient exhibits one or more symptoms of a neurodegenerative or demyelinating disorder, such as multiple sclerosis (MS), such as relapsing remitting MS (RRMS), secondary progressive MS (SPMS), primary progressive MS (PPMS), or progressive relapsing MS (PRMS). In some embodiments, the patient has clinically isolated syndrome (CIS), and is at risk of progressing to MS. In some embodiments, the patient has optic neuritis, neuromyelitis optica, or transverse myelitis. The invention can be applied to other autoimmune conditions as described herein.

In various embodiments, the methods involve determining an antibody repertoire of a patient, for example, by amplification and sequencing of antibody genes or transcripts from patient B cells. For example, antibody heavy chains (e.g., variable heavy chain sequences) can be sequenced and used to identify pathological sequences, or in some embodiments, paired heavy and light chains are sequenced. In some embodiments, antibodies of interest are cloned and evaluated by immunochemistry with samples that comprise the autoantigen (e.g., tissue, cell sample, or purified or partially purified antigen), to confirm that antibodies of interest are likely involved in the autoimmune pathology.

In some embodiments, the patient has a demyelinating disease such as MS, and mutation at defined codons in VH4 antibodies are evaluated, to identify the presence, absence, or relative level of VH4 antibodies that are indicative of MS or other demyelinating disease or inflammatory disease of the CNS. For example, VH4 antibodies having mutations in least 2 codons selected from 31B, 32, 40, 56, 57, 60, 81, and 89 (which correspond to progression to MS) are selected as being potentially pathological antibodies for the patient's disease.

Once antibody-encoding nucleotide sequences that correspond to the patient's autoimmune disease have been identified in the patient's antibody repertoire, the invention involves designing a therapeutic agent to target the identified sequences. In various embodiments, from 1 to about 20, or from 1 to about 10, or from 1 to about 5 (e.g., 1, 2, 3, 4, or 5) antibody sequences are identified for targeting. For example, the therapeutic agent(s) may be antisense oligonucleotides or siRNAs specific for one or more the sequences identified. In other embodiments, the therapeutic agents include one or more antibodies or fragments thereof, modified to lack immune effector functions relevant to the disease processes (e.g., inflammation and/or demyelination). The oligonucleotides and antibodies may be formulated as a variety of pharmaceutical compositions, including for administration by a variety of routes.

In other aspects, the invention provides methods for producing therapeutic antibodies to target destruction of unwanted cells, including malignant cells and pathogens. In these embodiments, B cells and corresponding antibodies are identified that can direct a productive or effective antibody response against the unwanted cells.

For example, in some embodiments a first population of B cells from one or more subjects displaying an immune response against a malignancy or infectious disease is provided, and the corresponding antibody genetics evaluated against antibodies from a second population of B cells that do not exhibit the immune response of interest. The second population of B cells can be from the same subjects as the first population, or from different subjects. For example, B cells can be isolated from tumor tissue or infected tissue of patients exhibiting a productive immune response, and resulting antibody genetics evaluated against antibody sequences from other B cell populations. The antibodies or antigen-binding fragments or portions thereof identified as involved in the immune response of interest, are cloned, recombinantly produced, and formulated for administration to patients.

All references cited herein are hereby incorporated by reference in their entirety.

Other aspects and embodiments of the present invention will be apparent from the following detailed description.

### DETAILED DESCRIPTION OF THE INVENTION

The present invention relates to treatment of disease, including treatment of antibody-mediated autoimmune disorders, as well as treatment of other conditions in which antibody therapeutics can be beneficial for targeting destruction of foreign or malignant cells.

In some aspects, to better inform and implement neuroprotective and neurodegenerative strategies, the present invention employs a biologically-directed approach to identify key pathophysiological antibody sequences and integrate them into a novel class of protein or oligonucleotide therapies. The present invention in various aspects provides therapy for demyelinating diseases, such as multiple sclerosis, without exposing the patient to the risk of a systemic immune cell ablation.

At present, there are no medications approved by the U.S. Food and Drug Administration (FDA) that provide neuroprotection in MS. The approved disease-modifying therapies work primarily by reducing inflammation in CNS, but are not well suited for a disease course that is characterized by nerve degeneration rather than inflammation. Preliminary results with Ocrelizumab suggest that B cell intervention can be a key strategy for the treatment of both RRMS and PPMS. However, Ocrelizumab destroys most of the B cells resident in a patient. Not surprisingly, this aggressive approach to disease intervention has clear limitations, as exhibited in earlier clinical trials for the treatment of Lupus and Rheumatoid Arthritis where the drug had to be abandoned because of an unacceptable number of serious and sometimes fatal opportunistic infections. A more precise therapy targeted to the pathological B cell component could drastically improve therapy for MS disease and related demyelinating conditions.

In various aspects, the invention provides methods for treating autoimmune diseases, such as demyelinatng diseases. In the various embodiments, the methods involve determining nucleotide sequences in a patient sample for a plurality of immunoglobulin-encoding genes or transcripts, and particularly the nucleotide sequences encoding at least a portion of the complementarity-determining region (CDR), which are representative of the antigen-binding specificity. By identifying nucleotide sequences that correspond to the autoimmune disease, oligonucleotide or protein therapeutics can be designed to specifically target the patient's pathological antibody component.

Exemplary antibody-mediated autoimmune diseases in which the invention finds use include multiple sclerosis, neuromyelitis optica, optic neuritis, transverse myelitis, acute disseminated encephalitis, systemic lupus erythematosus (SLE), rheumatoid arthritis, sjogren's disease, psoriasis, vasculitis, crohn's disease, and inflammatory bowel disease, among others. In some embodiments, the autoimmune disease is characterized by type II or type III hypersensitivity. In some embodiments, the autoimmune disease is a demyelinating disease.

In some embodiments, the patient exhibits one or more symptoms of a neurodegenerative or demyelinating disorder, such as multiple sclerosis (MS). In some embodiments, the patient may be diagnosed as having MS. MS is one of the most common diseases of the central nervous system (brain and spinal cord). It is an inflammatory condition associated with demyelination, or loss of the myelin sheath. Myelin, a fatty material that insulates nerves, acts as insulator in allowing nerves to transmit impulses from one point to another. In MS, the loss of myelin is accompanied by a disruption in the ability of the nerves to conduct electrical impulses to and from the brain and this produces the various symptoms of MS, such as impairments in vision, muscle coordination, strength, sensation, speech and swallowing, bladder control, sexuality and cognitive function. The plaques or lesions where myelin is lost appear as hardened, scar-like areas. These scars appear at different times and in different areas of the brain and spinal cord. In some embodiments, the patient has relapsing remitting MS (RRMS), secondary progressive MS (SPMS), primary progressive MS (PPMS), or progressive relapsing MS (PRMS).

In some embodiments, the patient has clinically isolated syndrome (CIS), and is at risk of progressing to MS. In some embodiments, the patient has optic neuritis, neuromyelitis optica, or transverse myelitis.

In various embodiments, the methods involve determining an antibody repertoire of a patient, for example, by amplification and sequencing of antibody genes or transcripts from patient B cells. Methods for determining an antibody repertoire by nucleotide sequencing have been described in US 2014/0371103 and US 2014/0357500, which are each hereby incorporated by reference in their entireties. Sequencing methods are generally in high-throughput, but can employ any sequencing platform including pyrosequencing, sequencing-by-synthesis, or nanopore sequencing platform, among others.

In various embodiments, antibody heavy chains or portions thereof preferably comprising one or more CDRs, can be sequenced and evaluated to identify pathological antibody sequences. In some embodiments, paired heavy and light chains are clonally amplified and sequenced. Sequencing can take place by any known sequencing process, including high throughput sequencing-by-synthesis or pyrosequencing. For example, without limitation, isolated immune cells can be encapsulated in water in oil emulsions to create individual picoliter compartments containing a single immune cell or less per droplet. Millions of cells can be processed for each patient allowing high throughput in single cell sequencing technology. Micron scale paramagnetic beads harboring oligonucleotides complementary to the VH and VL antibody chains are introduced during the emulsion process. These beads may carry long degenerate barcodes such that each bead can confer a unique identity code. The millions of single immune cells are lysed inside the emulsion and the antibody transcripts are reverse transcribed using the barcoded bead primers, followed by PCR amplification of the VH and VL chains. Each VH and VL chain stemming from a single immune cell can be virtually linked to each other with the same barcode identity.

The VH and VL chains are then recovered from the emulsion, and PCR enriched in order to add next-generation sequencing (NGS) tags. The library can be sequenced using a high throughput sequencing platform followed by analysis of repertoire diversity, antibody frequency, CDR3 characterization, somatic hypermutation phylogeny analysis, etc. A database of correctly matched VH and VL pairs can be generated by deconvoluting the bead barcode sequences. Because each single immune cells were isolated in their respective emulsion droplets, for each barcode observed twice, the transcripts sequenced originated from a same emulsion droplets and therefore from a unique single cell.

In parallel to the sequencing, select VH and VL sequences of interest can be optionally cloned into antibody expression vectors and co-transfected and produced for characterization. For example, antibodies of interest can be evaluated by immunochemistry with a source of autoantigen, which can be a tissue, cell or cell line, or purified or partially purified antigen. For example, in the case of MS or demyelinating disease, the source of autoantigen can be brain or CNS samples, and staining patterns can be evaluated for staining of white and gray matter. This process can confirm that the antibody is likely involved in disease pathology. In some embodiments, in the case of MS or demyelinating disease, the selected antibody binds an antigen in human and/or mouse gray matter.

In certain embodiments, antibody-producing immune cells can be isolated from the blood or other biological samples of the patient. In some embodiments, the lymphocyte pool can be enriched for the desired immune cells by any suitable method, such as screening and sorting the cells using fluorescence-activated cell sorting (FACS), magnetic activated cell sorting (MACS), panning or other screening method to generate a plurality of immune cells from a sample. In some embodiments, in the case of MS or demyelinating disease, B cells are recovered from a cerebrospinal fluid sample from the patient, or alternatively, the starting material is peripheral blood. The peripheral blood cells can be enriched for B cells or plasmablasts.

The germline VH genes are separated into at least six families (VH1 through VH6) based on DNA nucleotide sequence identity of the first 95 to 101 amino acids.

Members of the same family typically have 80% or more sequence identity, whereas members of different families have less than 70% identity. These families range in size from one VH6 gene to an estimated greater than 45 VH3 genes. For example, VH4 family genes, which are of interest for demyelinating diseases, contain 9 different members: 4-04, 4-28, 4-30, 4-31, 4-34, 4-39, 4-59, 4-61, 4-B.

Thus, in some embodiments, amplification of immune cell genetic material, e.g. reverse transcription polymerase chain reaction (RT-PCR), is employed to generate cDNA amplification of immune cell genetic material. For antibody molecules, the immunoglobulin genes can be obtained from genomic DNA or mRNA of immune cells. RNA can be heavy chain (V, D, J segments), or light chain (V, J segments), or portions thereof. In some embodiments, the starting material is RNA from immune cells composed of V, D, J gene segments.

While in some embodiments, the method amplifies and/or sequences IgG antibodies, one or more other isotypes can be sequenced alternatively or in addition, such as IgM, IgA, IgE and IgD isotypes, as well as antibody subtype. In the case of IgG, the sequencing can be specific for one or more of VH1, VH2, VH3, VH4, VH5, or VH6. In some embodiments, VH4 antibodies are sequenced, which are considered to play a role in the pathology of MS and related neurodegenerative diseases. Processes for sequencing VH4 sequences are disclosed in US 2014/0371103, which is hereby incorporated by reference.

In some embodiments, mutation at defined codons in VH4 antibodies are evaluated, to identify the presence, absence, or relative level of VH4 antibodies that are indicative of MS or other demyelinating disease or inflammatory disease of the CNS. For example, U.S. Pat. No. 8,394,583 (the entire disclosure of which is hereby incorporated by reference) describes a biomarker for conversion from CIS to clinically definite MS (CDMS) in the antibody genetics of V_{H}4-utilizing B cells. Antibodies having mutations in least 2 codons selected from 31B, 32, 40, 56, 57, 60, 81, and 89 (which correspond to progression to MS) are selected as being potentially pathological antibodies for the patient's disease. Codon positions are defined by Kabat numbering, and mutations are identified with respect to the germline sequence. VH4 genes or transcripts can include one or more (or all) subfamily genes, for example, 4-04, 4-28, 4-30, 4-31, 4-34, 4-39, 4-59, 4-61, and 4-B.

Further, VH4 signatures indicative of neuromyelitis optica (NMO) are described in WO 2013/059417, the entire contents of which are hereby incorporated by reference.

While it is not necessary to evaluate particular amino acid identities in various embodiments, exemplary substitutions at these codons are disclosed in WO 2015/070009, the entire disclosure of which is hereby incorporated by reference.

In various embodiments, signature VH4 sequences generally comprise two or more mutations at codon positions 31B, 32, 40, 56, 57, 60, 81, and 89. In various embodiments, the VH4 antibody comprises at least 3, or at least 4, or at least 5, or at least 6 mutations with respect to the germline sequence at these positions. In some embodiments, the VH4 antibody comprises mutations at one or more of codons 32, 40, 57, 60, and 89 with respect to the germline sequence. In some embodiments, the VH4 antibody comprises mutations at two or more of codons 56, 57, and 81 with respect to the germline sequence. In some embodiments, the VH4 antibody comprises mutations at two or more of codons 40, 56, 81, and 89 with respect to the germline sequence.

Various antibody clones that harbor the VH4 signature, and their immunohistochemistry staining patterns of brain tissue are disclosed in WO 2015/070009, which is hereby incorporated by reference in its entirety. For example, exemplary mutations at codon 31B include R, N, D, P, K, G, A, and T; exemplary mutations at codon 40 include S, L, and A; exemplary mutations at codon 56 include R, G, N, T, Y, H, D, and K; exemplary mutation at codon 57 include A, I, D, and S; exemplary mutations at codon 81 include N, R, and M; exemplary mutations at codon 89 include F, I, R, and L. The methods are however not limited to these mutations or sets of mutations described in WO 2015/070009.

Other antibody signatures that are indicative of other conditions can be identified by sequencing the antibody repertoire of a suitable patient cohort or isolated B cell population of interest, and comparing the sequences to a control B cell population. The control B cell population does not exhibit the reactivity of interest, and in various embodiments may be isolated from healthy controls, or controls that include other inflammatory or degenerative diseases, or from locations of the body not affected by the disease of interest. For example, antibody gene signatures can be identified using known classifier algorithms including, without limitation: Principal Components Analysis, Naive Bayes, Support Vector Machines, Nearest Neighbors, Decision Trees, Logistic, Artificial Neural Networks, Penalized Logistic Regression, and Rule-based schemes. In addition, the predictions from multiple models can be combined to generate an overall prediction. The process for preparing a suitable class predictor is reviewed in R. Simon, Diagnostic and prognostic prediction using gene expression profiles in high-dimensional microarray data, British Journal of Cancer (2003) 89, 1599-1604, which review is hereby incorporated by reference in its entirety.

Antibody signatures can include mutational frequency at select codons (usually in CDR regions of heavy and/or light chains), identity and/or frequency of amino acid substitutions (e.g., including analysis of conserved or non-conserved substitutions), antibody isotype, and V-D-J gene usage, for example.

Once signature nucleotide sequences that correspond to the autoimmune disease have been identified in the patient's antibody repertoire, the invention involves designing a therapeutic agent to target the identified antibody sequences. In various embodiments, from 1 to about 20, or from 1 to about 10, or from 1 to about 5 (e.g., 1, 2, 3, 4, or 5) sequences are identified for targeting. For example, the therapeutic agent may be one or more antisense oligonucleotides or siRNAs specific for one or more the sequences identified. In other embodiments, the therapeutic agent is a recombinant antibody or antigen-binding fragment or portion thereof.

Antisense oligonucleotides are typically in the range of 8 to 30 nucleotides in length, and 12 to 25 nucleotides in some embodiments, although the invention is not strictly tied to any particular size. Specifically, oligonucleotides can be designed that are complementary to the variable heavy or variable light sequences in regions harboring the antibody gene signature. For example, the oligonucleotide can be complementary to an antibody transcript in a region containing at least two codon mutations that are indicative of the disease. In some embodiments, the oligonucleotide is complementary to an antibody transcript in a region containing at least three or at least four codon mutations that are indicative of the disease. In some embodiments, antibody-encoding transcripts are identified that have at least 2, 3, or 4 mutations in the variable region that are indicative of disease, and which are within 20 nucleotides. For example, in some embodiments, a VH4 region of no more than about 25 or about 20 nucleotides, and within codons 31B to 89 of VH4, is identified that contains 2, 3, 4, or 5 (or more) signature mutations. These antibody-encoding transcripts are particularly suitable for antisense therapeutics. Oligonucleotide sequences are further identified that exhibit an appropriate Tm for therapeutic application, which can be further adjusted by nucleotide modifications are further described below.

In some embodiments, the same transcript is targeted by at least two antisense oligonucleotides, to thereby target additional regions harboring the antibody gene signature.

In some embodiments, siRNA(s) are designed to inhibit expression of the identified gene sequences. Structure, formulation, and delivery of siRNAs are described, for example, in US 2014/0161894, US 2014/0024699, US 2015/0197746, and US 2016/0076040, which are hereby incorporated by reference in their entireties. See also, Fakhr E. et al., Precise and efficient siRNA design: a key point in competent gene silencing. Cancer Gene Ther. 2016 April; 23(4):73-82. Generally, design of siRNAs includes analysis of factors such as distance of target region to transcription start site, nucleotide composition, potential for off-target effects, secondary structures in the target site and siRNA, and the presence of asymmetry and energy valley within the siRNA.

Other methods and algorithms for designing oligonucleotides such as antisense oligonucleotides or siRNAs are known in the art. For example, modeling programs such as Foldsplit may be used to design antisense oligonucleotides (see Sczakiel G., et al (1993) Computer-aided search for effective antisense RNA target sequences of the human immunodeficiency virus type 1. Antisense Res. Dev. 3, 45-52). Algorithms for designing siRNAs are also known and include, without limitation, the Whitehead siRNA Selection Web Server (http://jura.wi.mit.edu/bioc/siRNA) as well as others described in Boese Q. et al., (2005) Mechanistic insights aid computational short interfering RNA design. Methods Enzymol. 392:73-96.

Oligonucleotide therapeutics may contain one or more chemical modifications to impart stability and/or increase affinity for the target site, among other advantages. Exemplary chemical modifications include backbone modifications (e.g., phosphorothioate or phosphorodiamidate morpholino), as well as 2′ modifications (e.g., 2′ O-methyl or 2′ halo) and bridging modifications (e.g., locked nucleic acid, or other 2′ to 4′ bridge structure), base modifications, and/or cap structures. Exemplary modifications are described in U.S. Pat. No. 9,163,235, U.S. Pat. No. 8,642,751, and WO 2012/061810, which are hereby incorporated by reference in their entireties.

The oligonucleotides may include at least one locked nucleotide (LNA), as described, for example, in U.S. Pat. Nos. 6,268,490, 6,316,198, 6,403,566, 6,770,748, 6,998,484, 6,670,461, and 7,034,133, all of which are hereby incorporated by reference in their entireties.

In certain embodiments, the oligonucleotide further comprises at least one terminal modification or “cap”. The cap may be a 5′ and/or a 3′-cap structure. The terms “cap” or “end-cap” include chemical modifications at either terminus of the oligonucleotide (with respect to terminal ribonucleotides), and including modifications at the linkage between the last two nucleotides on the 5′ end and the last two nucleotides on the 3′ end. The cap structure may increase resistance of the oligonucleotide to exonucleases without compromising molecular interactions with the RNA target. Such modifications may be selected on the basis of their increased potency in vitro or in vivo. The cap can be present at the 5′-terminus (5′-cap) or at the 3′-terminus (3′-cap) or can be present on both ends. In certain embodiments, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic moiety (2′-3′ or 3′-3′), phosphorodithioate monophosphate, and methylphosphonate moiety.

The oligonucleotide may contain one or more phosphorothioate linkages. Phosphorothioate linkages have been used to render oligonucleotides more resistant to nuclease cleavage. For example, the polynucleotide may be partially phosphorothioate-linked, for example, phosphorothioate linkages may alternate with phosphodiester linkages. In certain embodiments, however, the oligonucleotide is fully phosphorothioate-linked. In other embodiments, the oligonucleotide has from one to five or one to three phosphate linkages.

In a further embodiment, the oligonucleotide may include one or more morpholino nucleotides. In another embodiment, the oligonucleotide may include one or more peptide nucleic acids (PNAs), which contain a peptide-based backbone rather than a sugar-phosphate backbone.

The oligonucleotides may be produced by a variety of techniques known in the art. For example, the synthesis of oligonucleotides, including modified polynucleotides, by solid phase synthesis is well known and is reviewed by Caruthers et al., New Chemical Methods for Synthesizing Polynucleotides, Nucleic Acids Symp. Ser., (7):215-23 (1980) which is hereby incorporated by reference in its entirety. The oligonucleotides, for example, siRNA, may also be synthesized by in vitro transcription (e.g., T7, T3 and/or SP6 RNA polymerase transcription with or without processing by ribonucleases such as T1 ribonuclease, or processing by phosphatases) or recombinant human Dicer/E. coli RNaseIII digestion of long double-stranded RNA molecules.

The oligonucleotide may be incorporated within a variety of macromolecular assemblies or compositions. Such complexes for delivery may include a variety of liposomes, nanoparticles, and micelles, formulated for delivery to a patient. In some embodiments, the oligonucleotides are incorporated within nanoparticles. For example, the oligonucleotides may be complexed with lipids thus forming lipid nanoparticles (LNPs). In another embodiment, the oligonucleotides may be complexed with polymeric nanocarriers to form polymeric nanoparticles. Polymeric nanoparticles may be formed from PLA or PLGA, PLA-PEG, PLGA-PEG, or combinations thereof

In various embodiments, the complexes may include one or more fusogenic or lipophilic molecules to initiate cellular membrane penetration. Such molecules are described, for example, in U.S. Pat. No. 7,404,969 and U.S. Pat. No. 7,202,227, which are hereby incorporated by reference in their entireties. Alternatively, the oligonucleotide may further comprise a pendant lipophilic group to aid cellular delivery, such as those described in WO 2010/129672, which is hereby incorporated by reference. In various embodiments, the oligonucleotides are conjugated to peptides, antibodies, polymers, ligands, and/or aptamers for targeted delivery. For example, the oligonucleotides may be conjugated to positively charged polymers. Examples of positively charged polymers include peptides, such as arginine rich peptides or TAT sequence. Another example of positively charged polymers is polyethylenimine (PEI) with multiple positively charged amine groups in its branched or unbranched chains.

In an embodiment, the oligonucleotides or complexes comprising the same are targeted for delivery to B cells. For example, the oligonucleotides may be associated with a B-cell targeting ligand. In another example, the oligonucleotides or complexes comprising the same may be associated with an antibody that recognizes a B-cell marker such as CD10, CD19, CD20, CD21, CD22, CD23, CD24, CD37, CD38, CD39, CD40, CD70, CD72, CD73, CD74, CDw75, CDw76, CD77, CD78, CD79a/b, CD80, CD81, CD82, CD83, CD84, CD85, CD86, CD89, CD98, CD126, CD127, CDw130, CD138, or CDw150.

The pharmaceutical compositions comprising an effective amount of the oligonucleotide(s) further comprise a pharmaceutically-acceptable carrier or diluent.

The composition or formulation may employ a plurality of therapeutic oligonucleotides, including at least one described herein. For example, the composition or formulation may employ at least about 2, about 3, about 4, about 5, about 6, about 7, about 8, about 9, or about 10 oligonucleotides according to the present disclosure. In some embodiments, the compositions may include one or more oligonucleotides targeting the signature VH4 sequences unique to a patient.

The oligonucleotides of the invention may be formulated as a variety of pharmaceutical compositions. Pharmaceutical compositions will be prepared in a form appropriate for the intended application. Generally, this entails preparing compositions that are essentially free of pyrogens, as well as other impurities that could be harmful to humans or animals. Exemplary delivery/formulation systems include colloidal dispersion systems, macromolecule complexes, nanocapsules, microspheres, beads, and lipid-based systems including oil-in-water emulsions, micelles, mixed micelles, and liposomes. Commercially available fat emulsions that are suitable for delivering the nucleic acids of the invention include Intralipid®, Liposyn®, Liposyn® II, Liposyn® III, Nutrilipid, and other similar lipid emulsions. An exemplary colloidal system for use as a delivery vehicle in vivo is a liposome (i.e., an artificial membrane vesicle). The preparation and use of such systems is well known in the art. Exemplary formulations are also disclosed in U.S. Pat. No. 5,981,505; U.S. Pat. No. 6,217,900; U.S. Pat. No. 6,383,512; U.S. Pat. No. 5,783,565; U.S. Pat. No. 7,202,227; U.S. Pat. No. 6,379,965; U.S. Pat. No. 6,127,170; U.S. Pat. No. 5,837,533; U.S. Pat. No. 6,747,014; and WO 2003/093449, which are hereby incorporated by reference in their entireties.

The pharmaceutical compositions and formulations may employ appropriate salts and buffers to render delivery vehicles stable and allow for uptake by target cells. Aqueous compositions comprise an effective amount of the delivery vehicle comprising the oligonucleotide (e.g. liposomes or other complexes), dissolved or dispersed in a pharmaceutically acceptable carrier or aqueous medium. As used herein, “pharmaceutically acceptable carrier” may include one or more solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Supplementary active ingredients also can be incorporated into the compositions.

In various embodiments, the present invention further relates to synthesizing one or more antibodies or antigen-binding fragments thereof encoded by identified antibody sequences indicative of the patient's disease. In some embodiments, the antibodies are VH4 antibodies. In some embodiments, the antibodies or antigen-binding fragments are cloned from the identified sequences, and expressed and recovered from a cell culture system. The antibodies, generally lacking a functional Fc, are delivered to a patient to reduce or eliminate symptoms of the autoimmune or demyelinating disease, for example, by blocking the interaction of the endogenous autoantibodies with target tissue. Alternatively, or in addition, the present invention provides antibodies or anti-binding fragments thereof that bind to and neutralize the autoantibodies comprising the signature antibody sequences, thereby reducing or eliminating symptoms of the autoimmune disease.

In various embodiments, one or more antibody fragment or antigen-binding molecules are prepared against the identified antibodies. Each heavy chain includes one variable region (e.g., VH) and at least three constant regions (e.g., CH1, CH2 and CH3), and each light chain includes one variable region (VL) and one constant region (CL). The variable regions determine the specificity of the antibody. Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity. In some embodiments, the antibody is an antigen-binding fragment based on one or more identified antibody-encoding sequences. In an embodiment, the antibody or antigen-binding fragments thereof lack an Fc domain. In some embodiments, the antigen-binding portion thereof is incorporated into a bispecific antibody to combine other functionalities.

In some embodiments, the present invention provides for preparation of the antibody or antigen-binding molecule using any known platform, such as a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; a Microbody; a peptide aptamer; an alterases; a plastic antibodies; a phylomer; a stradobodies; a maxibodies; an evibody; a fynomer, an armadillo repeat protein, a Kunitz domain, an avimer, an atrimer, a probody, an immunobody, a triomab, a troybody; a pepbody; a vaccibody, a UniBody; affimers, a DuoBody, a Fv, a Fab, a Fab′, a F(ab′)2, a peptide mimetic molecule, or a synthetic molecule, as described in U.S. Pat. Nos. or Patent Publication Nos. U.S. Pat. No. 7,417,130, US 2004/132094, U.S. Pat. No. 5,831,012, US 2004/023334, U.S. Pat. No. 7,250,297, U.S. Pat. No. 6,818,418, US 2004/209243, U.S. Pat. No. 7,838,629, U.S. Pat. No. 7,186,524, U.S. Pat. No. 6,004,746, U.S. Pat. No. 5,475,096, US 2004/146938, US 2004/157209, U.S. Pat. No. 6,994,982, U.S. Pat. No. 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, antibody fragments are prepared having the specificity of the full-length antibody comprising the identified sequences, but lacking effector functions (e.g., lacking Fc structures). The Fc region interacts with various cell surface receptors such as the Fc receptors and complement proteins thereby allowing the antibody to activate the immune system. Without wishing to be bound by theory, it is believed that administration of antibody fragments that lack the Fc structures (e.g., Fab, Fab′, F(ab′)₂) or mutations that reduce or eliminate Fc receptor binding, provide targeted immune-suppressive effects to block immune processes involved in MS or other demyelinating or autoimmune disease.

The antibody in various embodiments is a human recombinant antibody. Antibodies may be produced by standard methods well known in the art (see, e.g., Antibodies: A Laboratory Manual, Cold Spring Harbor Laboratory, 1988; U.S. Pat. No. 4,196,265). For example, the VH4 antibodies can be prepared by co-transfection of cells with paired cloning vectors harboring IgK and IgH genes of the desired VH4 antibody. Recombinant antibodies can be harvested from transfected cell supernatants.

Methods for producing the antibodies or antigen-binding fragments thereof are known in the art. For example, DNA sequences encoding the antibodies or antigen-binding fragments thereof can be cloned or chemically synthesized using methods known in the art. Synthetic DNA sequences can be ligated to other appropriate nucleotide sequences, including, e.g., expression control sequences, to produce gene expression constructs encoding the desired antibodies or antigen-binding fragments thereof.

Nucleic acids encoding the antibodies or antigen-binding fragments thereof can be incorporated (ligated) into expression vectors, which can be introduced into host cells through transfection, transformation, or transduction techniques. For example, nucleic acids encoding the antibodies or antigen-binding fragments thereof can be introduced into host cells by retroviral transduction. Illustrative host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the antibodies or antigen-binding fragments thereof. Accordingly, in some embodiments, the present invention provides for a nucleic acid encoding an antibody or an antigen-binding fragment thereof. In some embodiments, the present invention provides expression vectors comprising nucleic acids that encode the antibodies or antigen-binding fragments thereof In some embodiments, the present invention provides for a host cell comprising the nucleic acid encoding an antibody or an antigen-binding fragment thereof. In some embodiments, the present invention additional provides host cells comprising the expression vectors of the invention.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. In another example, if the engineered gene is to be expressed in eukaryotic host cells, e.g., CHO cells, it is first inserted into an expression vector containing for example, a suitable eukaryotic promoter, a secretion signal, enhancers, and various introns. The gene construct can be introduced into the host cells using transfection, transformation, or transduction techniques.

The antibodies or antigen-binding fragments thereof can be produced by growing a host cell transfected with an expression vector encoding the antibodies or antigen-binding fragments thereof under conditions that permit expression of the protein. Following expression, the protein can be harvested and purified using techniques well known in the art, e.g., affinity tags such as glutathione-S-transferase (GST) and histidine tags or by chromatography. Antibody fragments can be prepared by know processes, including a Fab, Fab′, or F(ab′)₂ fragments, for delivery to the patient.

Accordingly, the present invention further provides pharmaceutical compositions that comprise an effective amount of the antibodies or antigen-binding fragments thereof or pharmaceutically-acceptable salt thereof, and a pharmaceutically-acceptable carrier or diluent. Methods for formulating such compositions are described previously.

In various embodiments, any pharmaceutical composition described herein is formulated in accordance with routine procedures as a composition adapted for a mode of administration described herein. For example, any pharmaceutical composition described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use.

Exemplary routes of administration include, for example: oral, intradermal, intrathecal, intramuscular, intraperitoneal, intravenous, subcutaneous, intranasal, epidural, sublingual, intranasal, intracerebral, intravaginal, transdermal, rectally, by inhalation, or topically. Administration can be local or systemic. The mode of administration can be left to the discretion of the practitioner, and will depend in-part upon the site of the medical condition. In some embodiments, the pharmaceutical composition is formulated for parenteral administration (e.g. intravenous, intramuscular, intraperitoneal, subcutaneous, intrathecal, and infusion).

Formulations suitable for parenteral administration include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

In various embodiments, the present invention is used to treat MS. In various embodiments, the therapeutic agents described herein (e.g., oligonucleotide-based or protein based therapeutic agents) are used to eliminate and reduce multiple MS symptoms. Illustrative symptoms associated with multiple sclerosis, which can be potentially ameliorated or prevented with the compositions and methods described herein, include: optic neuritis, diplopia, nystagmus, ocular dysmetria, internuclear ophthalmoplegia, movement and sound phosphenes, afferent pupillary defect, paresis, monoparesis, paraparesis, hemiparesis, quadraparesis, plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop, dysfunctional reflexes, paresthesia, anesthesia, neuralgia, neuropathic and neurogenic pain, Lhermitte's sign, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, frigidity, constipation, fecal urgency, fecal incontinence, depression, cognitive dysfunction, dementia, mood swings, emotional lability, euphoria, bipolar syndrome, anxiety, aphasia, dysphasia, fatigue, Uhthoffs symptom, gastroesophageal reflux, and sleeping disorders.

In various embodiments, the present methods are used to treat clinically isolated syndrome (CIS). A clinically isolated syndrome (CIS) is a single monosymptomatic attack compatible with MS, such as optic neuritis, brain stem symptoms, and partial myelitis. Patients with CIS that experience a second clinical attack are generally considered to have clinically definite multiple sclerosis (CDMS). Over 80 percent of patients with CIS and MRI lesions go on to develop MS, while approximately 20 percent have a self-limited process. Patients who experience a single clinical attack consistent with MS may have at least one lesion consistent with multiple sclerosis prior to the development of clinically definite multiple sclerosis. In various embodiments, the present methods are used to treat CIS so it does not develop into MS.

In various embodiments, the present methods are used to treat radiologically isolated syndrome (RIS). In RIS, incidental imaging findings suggest inflammatory demyelination in the absence of clinical signs or symptoms. In various embodiments, the present methods are used to treat RIS so it does not develop into MS.

In various embodiments, the present oligonucleotide or protein-based agents are used to treat benign multiple sclerosis; relapsing-remitting multiple sclerosis (RRMS); secondary progressive multiple sclerosis (SPMS); progressive relapsing multiple sclerosis (PRMS); and primary progressive multiple sclerosis (PPMS).

Benign multiple sclerosis is a retrospective diagnosis which is characterized by 1-2 exacerbations with complete recovery, no lasting disability and no disease progression for 10-15 years after the initial onset. Benign multiple sclerosis may, however, progress into other forms of multiple sclerosis. In various embodiments, the present methods are used to treat benign multiple sclerosis so it does not develop into MS.

Patients suffering from RRMS experience sporadic exacerbations or relapses, as well as periods of remission. Lesions and evidence of axonal loss may or may not be visible on MRI for patients with RRMS. In various embodiments, the present methods are used to treat RRMS. A clinical relapse, which may also be used herein as “relapse,” “confirmed relapse,” or “clinically defined relapse,” is the appearance of one or more new neurological abnormalities or the reappearance of one or more previously observed neurological abnormalities.

SPMS may evolve from RRMS. Patients afflicted with SPMS have relapses, a diminishing degree of recovery during remissions, less frequent remissions and more pronounced neurological deficits than RRMS patients. Enlarged ventricles, which are markers for atrophy of the corpus callosum, midline center and spinal cord, are visible on MRI of patients with SPMS. In various embodiments, the present methods are used to treat RRMS so it does not develop into SPMS.

PPMS is characterized by a steady progression of increasing neurological deficits without distinct attacks or remissions. Cerebral lesions, diffuse spinal cord damage and evidence of axonal loss are evident on the MRI of patients with PPMS. PPMS has periods of acute exacerbations while proceeding along a course of increasing neurological deficits without remissions. Lesions are evident on MRI of patients suffering from PRMS. In various embodiments, the present methods are used to treat RRMS and/or SPMS so it does not develop into PPMS.

In some embodiments, the present methods are used to treat relapsing forms of MS. In some embodiments, the present methods are used to treat relapsing forms of MS to slow the accumulation of physical disability and/or reduce the frequency of clinical exacerbations, and, optionally, for patients who have experienced a first clinical episode and have MRI features consistent with MS. In some embodiments, the present methods are used to treat worsening relapsing-remitting MS, progressive-relapsing MS or secondary-progressive MS to reduce neurologic disability and/or the frequency of clinical exacerbations. In some embodiments, the present methods can effectively reduce the frequency and/or severity of relapses.

In some embodiments, various clinical or other indicia of effectiveness of treatment may be used, e.g., expanded disability status score (EDSS); MRI scan; relapse number, rate, or severity; multiple sclerosis functional composite (MSFC); multiple sclerosis quality of life inventory (MSQLI); Paced Serial Addition Test (PASAT); symbol digit modalities test (SDMT); 25-foot walk test; 9-hole peg test; low contrast visual acuity; Modified Fatigue Impact Scale; multiple sclerosis functional composite (MSFC); Beck Depression Inventory; and 7/24 Spatial Recall Test can be used. In some embodiments, the present methods cause an improvement in one or more of these measures.

In some embodiments, the oligonucleotides, antibodies or antigen-binding fragments thereof, or pharmaceutical compositions are administered to a patient undergoing treatment with another therapeutic agent. In some embodiments, the oligonucleotides, antibodies or antigen-binding fragments thereof, or pharmaceutical compositions of the invention are administered to a patient before, during, or after treatment with another therapeutic agent. In some embodiments, the other therapeutic agent is part of a disease-modifying therapy for treating MS. Exemplary MS disease-modifying therapeutic agents include, but are not limited to, one or more of teriflunomide (AUBAGIO (Genzyme)); interferon beta-1a (AVONEX (Biogen Idec); interferon beta-1b (BETASERON (Bayer Healthcare Pharmaceuticals, Inc.); glatiramer acetate (COPAXONE (Teva Neuroscience); interferon beta-1b (EXTAVIA (Novartis Pharmaceuticals Corp.); fingolimod (GILENYA (Novartis Pharmaceuticals Corp.); alemtuzumab (LEMTRADA (Genzyme); mitoxantrone (NOVANTRONE (EMD Serono, Inc.); pegylated interferon beta-1a (PLEGRIDY (BIOGEN IDEC); interferon beta-1a (REBIF (EMD Serono, Inc.)); dimethyl fumarate (BG-12) (TECFIDERA (Biogen Idec); and natalizumab (TYSABRI (Biogen Idec).

In other aspects, the invention provides methods for producing therapeutic antibodies to target destruction of unwanted cells, including malignant cells and pathogens. In these embodiments, B cells and corresponding antibodies are identified that can direct a productive or effective antibody response.

For example, in some cases of solid tumors, evidence of inflammation (i.e. lymphocyte infiltration) within or near the tumor is predictive of a positive immune response. B cells may be participating in this immune response by generating specific antibodies targeting the tumor for destruction. These antibodies targeting the tumor could be of either a particular heavy or light chain gene family, or carry particular mutations within their heavy or light chain genes. Thus, antibody genes isolated from single B cells identified within or near the tumor can be cloned and tested for their ability to bind to tumor antigens. Antibodies that bind to tumor antigens are binned separate from those that do not, and the antibody genetics of the two groups compared. For individual patients, this would provide an avenue for producing one or more of the specific antibodies ex vivo and giving it back to the patient as a therapeutic to more effectively target the tumor for destruction, or even use as an imaging enhancer. Further, the genetics of tumor binding antibodies for several patients with the same tumor type can be determined, with the hypothesis that there are particular heavy or light chain genes or particular mutations within the heavy and light chain genes of tumor-binding antibodies that are common to all (or many) patients with a particular tumor type. These antibodies could also be produced ex vivo and given to patients with this type of tumor, whether they make that particular antibody or not. Similar strategies can be effective for various infectious diseases, where antibodies that target pathogen or pathogen-associated molecules are needed.

Thus, in some embodiments, the invention provides a method for producing a therapeutic antibody. The method comprises providing a first population of B cells from one or more subjects displaying an immune response against a malignancy or infectious disease, and a second population of B cells that do not display said immune response. The presence of desired binding to target cells (e.g., displayed by the first population, but not the second), can be determined by standard immunochemistry methods. The method further comprises determining nucleotide sequences for immunoglobulin-encoding genes or transcripts in the populations, and identifying immunoglobulin-encoding sequences associated with the immune response (e.g., binding to target cells). The method further comprises producing antibodies or antigen-binding portions thereof encoded by one or more of the nucleotide sequences that are associated with the immune response. Antigen-binding affinity can be confirmed with the recombinantly produced antibodies.

In some embodiments, the B cells displaying an immune response are from a patient having a malignancy. Exemplary malignancies include squamous or basal cell carcinoma, melanoma, biliary tract cancer, bladder cancer, bone cancer, brain or central nervous system cancer, breast cancer, cancer of the peritoneum, cervical cancer, colon or rectum cancer, cancer of the digestive system, endometrial cancer, esophageal cancer, eye cancer, cancer of the head and neck, gastric cancer, glioblastoma, neuroblastoma, liver cancer, kidney cancer, larynx cancer, leukemia, lung cancer, small-cell lung cancer, non-small cell lung cancer, adenocarcinoma of the lung, mesothelioma, myeloma, oral cavity cancer, ovarian cancer, pancreatic cancer, prostate cancer, retinoblastoma, rhabdomyosarcoma, salivary gland carcinoma, testicular cancer, thyroid cancer, uterine cancer, lymphoma, Hodgkin's lymphoma, non-Hodgkin's lymphoma, Cutaneous T-Cell Lymphoma, or B-cell lymphoma.

In some embodiments, the malignancy is a solid tumor. B cells are optionally isolated from tumor tissue or surrounding tissue, or in some embodiments are isolated from peripheral blood.

The first population of B cells can be distinguished empirically from the second population of B cells by the affinity of the antibody produced for tumor cells or tumor antigens. The second population of B cells (the control population) can be from the same patient(s) as the first population, but isolated from non-tumor tissue, such as peripheral blood in some embodiments. The second population in some embodiments comprises B cells from patients who have a different tumor type or are non-cancerous, or in some embodiments are isolated from patients with the tumor type, but who are not mounting an effective antibody response.

Antibody gene sequencing, evaluation of antibody genetics, and cloning can be conducted as discussed elsewhere herein in the context of autoimmune disease. The antibodies or antigen-binding portions thereof are recombinantly produced and administered to patients to target the tumor for destruction or as an imaging reagent (e.g., with conjugated detectable label).

In some embodiments, the subject has an infectious disease, which may be caused by a bacteria, virus, fungus, or parasite. For example, B cells are isolated from infected tissue, or are isolated from peripheral blood or other suitable source. The first population of B cells can be distinguished from the second population of B cells by their affinity for pathogen cells or pathogen-associated antigens, and this process can employ automated techniques using immobilized antigen in some embodiments. In some embodiments, the second population of B cells are from the same patient(s) as the first population, but are isolated from non-infected tissue or peripheral blood in some embodiments. In other embodiments, the second population comprises B cells from patients that do not display a productive immune response against the same pathogen, or are isolated from patients testing positive for a different (but optionally related) pathogen or non-pathogenic microbe. In some embodiments, the second population are isolated from healthy controls.

The antibodies or antigen-binding portions thereof are recombinantly produced and administered to one or more patients to target the pathogen.

In various embodiments, the pathogen may be a virus. Exemplary viruses include retroviruses (e.g., HIV), herpes simplex viruses (e.g, HSV-1, HSV-2, or varicella zoster), hepatitis viruses (e.g., HAV, HBV, HCV), adenovirus, HPV, Epstein-Barr Virus (EBV), Ebola, Marburg virus, and zika virus.

In some embodiments, the infectious disease is a persistent or recurrent bacterial infection, such as that associated with pneumonia, bronchitis, sinusitis, vaginitis, enteritis, colitis, sepsis, or urinary tract infection. Exemplary bacterial pathogens for which the invention may be effective include species of Mycobacterium (including tuberculosis), Pseudomonas (e.g., Pseudomonas aeruginosa, as may occur in association with cystic fibrosis), Haemophilus (e.g., Haemophilus influenzae), Moraxella, Chlamydia, Neisseria, Streptococcus, Staphylococcus (including MRSA), Bordetella, Yersinia, and others.

Exemplary fungal or parasitic infections include Candidiasis (e.g., yeast vaginitis), malaria, trypanosomiasis, Aspergillus infection, toxoplasma, and Giardiasis.

Modifications of the disclosed embodiments incorporating the spirit and substance of the invention may occur to persons skilled in the art and such modifications are within the scope of the present invention.

All references cited herein are hereby incorporated by reference in their entirety.

1. A method for treating an antibody-mediated autoimmune disease, the method comprising:
determining nucleotide sequences in a patient sample for a plurality of immunoglobulin-encoding genes or transcripts, the nucleotide sequences encoding at least a portion of the complementarity-determining region (CDR);
identifying one or more nucleotide sequences that correspond to the autoimmune disease;
synthesizing one or more oligonucleotides to reduce or inhibit the expression of said one or more nucleotide sequences; and
administering the one or more oligonucleotides to said patient.

2. The method of claim 1, wherein the antibody-mediated autoimmune disease is multiple sclerosis, neuromyelitis optica, optic neuritis, transverse myelitis, acute disseminated encephalitis, systemic lupus erythematosus (SLE), rheumatoid arthritis, sjogren's disease, psoriasis, vasculitis, crohn's disease, or inflammatory bowel disease.

3. The method of claim 1, wherein the antibody-mediated autoimmune disease is characterized by type II or type III hypersensitivity.

4. The method of claim 1, wherein the antibody mediated autoimmune disease is a demyelinating disease.

5. The method of claim 4, wherein the immunoglobulin-encoding genes or transcripts are VH4 genes or transcripts.

6. The method of claim 5, wherein the VH4 gene or transcript has at least two mutations with respect to the germline sequence at codons selected from 31B, 32, 40, 56, 57, 60, 81, and 89.

7. The method of claim 6, wherein the VH4 antibody binds to an antigen in human gray matter.

8. The method of claim 6 or 7, wherein the VH4 antibody has 3, 4, 5, or 6 mutations with respect to the germline sequence at codons selected from 31B, 32, 40, 56, 57, 60, 81, and 89.

9. The method of claim 8, wherein the VH4 antibody has mutations at two or more of codons 56, 57, and 81 with respect to the germline sequence, or at two or more of codons 40, 56, 81, and 89 with respect to the germline sequence.

10. The method of claim 8, wherein the VH4 antibody has mutations at one or more of codons 32, 40, 57, 60, and 89 with respect to the germline sequence.

11. The method of any one of claims 6 to 10, wherein the VH4 antibody has one or more of the following mutations:
codon 31B is R, N, D, P, K, G, A, or T, or is selected from R, N, and D;
codon 40 is S, L, or A;
codon 56 is selected from R, G, N, T, Y, H, D, and K, or is selected from N, T, and G;
codon 57 is A, I, D, S, or K, or is selected from A and I;
codon 81 is N, R, or M; and
codon 89 is selected from F, I, R, and L.

12. The method of claim 11, wherein the VH4 antibody has an R or N replacement at codon 81.

13. The method of claim 11, wherein the VH4 antibody has a T or R replacement at codon 56, optionally with the germline amino acid at codons 31B, 40, and 89, and which optionally binds astrocytes.

14. The method of any one of claims 5 to 13, wherein the VH4 germline is 4-04, 4-28, 4-30, 4-31, 4-34, 4-39, 4-59, 4-61 or 4-B.

15. The method of any one of claims 6 to 14, wherein the antibody has a set of mutations selected from Table 1.

16. The method of any one of claims 1 to 15, wherein the neurodegenerative disease is multiple sclerosis (MS).

17. The method of any one of claims 1 to 16, wherein the oligonucleotide is an antisense oligonucleotide.

18. The method of any one of claims 1 to 16, wherein the oligonucleotide is an siRNA.

19. The method of claim 17 or 18, wherein the oligonucleotide comprises one or more modifications selected from a locked nucleic acid, 2′ nucleotide modification, back-bone modification, or base modification.

20. The method of any one of claims 1 to 19, wherein the oligonucleotide is formulated for administration by subcutaneous, intravenous, intramuscular, oral, or intrathecal administration.

21. The method of any one of claims 1 to 20, wherein the oligonucleotide is associated with a B-cell targeting ligand.

22. The method of claim 21, wherein the oligonucleotide is formulated in nanoparticles.

23. A method for making an active agent for treating an antibody-mediated autoimmune disease, the method comprising:
determining nucleotide sequences in a patient sample for a plurality of immunoglobulin-encoding genes or transcripts, the nucleotide sequences encoding at least a portion of the complementarity-determining region (CDR);
identifying one or more nucleotide sequences that correspond to the autoimmune disease;
synthesizing one or more oligonucleotides to reduce or inhibit the expression of said one or more nucleotide sequences; and
formulating the one or more oligonucleotides for administration to said patient.

24. The method of claim 23, wherein the antibody-mediated autoimmune disease is multiple sclerosis, neuromyelitis optica, optic neuritis, transverse myelitis, acute disseminated encephalitis, systemic lupus erythematosus (SLE), rheumatoid arthritis, sjogren's disease, psoriasis, vasculitis, crohn's disease, or inflammatory bowel disease.

25. The method of claim 24, wherein the antibody-mediated autoimmune disease is characterized by type II or type III hypersensitivity.

26. The method of claim 23, wherein the antibody-mediated autoimmune disease is a demyelinating disease.

27. The method of claim 26, wherein the immunoglobulin-encoding genes or transcripts are VH4 genes or transcripts.

28. The method of claim 27, wherein the VH4 gene or transcript has at least two mutations with respect to the germline sequence at codons selected from 31B, 32, 40, 56, 57, 60, 81, and 89.

29. The method of claim 28, wherein the VH4 antibody binds to an antigen in human gray matter.

30. The method of claim 28 or 29, wherein the VH4 antibody has 3, 4, 5, or 6 mutations with respect to the germline sequence at codons selected from 31B, 32, 40, 56, 57, 60, 81, and 89.

31. The method of claim 30, wherein the VH4 antibody has mutations at two or more of codons 56, 57, and 81 with respect to the germline sequence, or at two or more of codons 40, 56, 81, and 89 with respect to the germline sequence.

32. The method of claim 30, wherein the VH4 antibody has mutations at one or more of codons 32, 40, 57, 60, and 89 with respect to the germline sequence.

33. The method of any one of claims 28 to 32, wherein the VH4 antibody has one or more of the following mutations:
codon 31B is R, N, D, P, K, G, A, or T, or is selected from R, N, and D;
codon 40 is S, L, or A;
codon 56 is selected from R, G, N, T, Y, H, D, and K, or is selected from N, T, and G;
codon 57 is A, I, D, S, or K, or is selected from A and I;
codon 81 is N, R, or M; and
codon 89 is selected from F, I, R, and L.

34. The method of claim 33, wherein the VH4 antibody has an R or N replacement at codon 81.

35. The method of claim 33, wherein the VH4 antibody has a T or R replacement at codon 56, optionally with the germline amino acid at codons 31B, 40, and 89, and which optionally binds astrocytes.

36. The method of any one of claims 27 to 35, wherein the VH4 germline is 4-04, 4-28, 4-30, 4-31, 4-34, 4-39, 4-59, 4-61 or 4-B.

37. The method of any one of claims 28 to 36, wherein the antibody has a set of mutations selected from Table 1.

38. The method of any one of claims 26 to 37, wherein the demyelinating disease is multiple sclerosis (MS).

39. The method of any one of claims 26 to 38, wherein the oligonucleotide is an antisense oligonucleotide.

40. The method of any one of claims 26 to 38, wherein the oligonucleotide is an siRNA.

41. The method of claim 39 or 40, wherein the oligonucleotide comprises one or more modifications selected from a locked nucleic acid, 2′ nucleotide modification, back-bone modification, or base modification.

42. The method of any one of claims 26 to 41, wherein the oligonucleotide is formulated for administration by subcutaneous, intravenous, intramuscular, oral, or intrathecal administration.

43. The method of any one of claims 26 to 42, wherein the oligonucleotide is associated with a B-cell targeting ligand.

44. The method of claim 43, wherein the oligonucleotide is formulated in nanoparticles.

45. A pharmaceutical formulation prepared by the method of any one of claims 26 to 44.

46. A method for treating an antibody-mediated autoimmune disease, the process comprising:
determining nucleotide sequences in a patient sample for a plurality of immunoglobulin-encoding genes or transcripts;
identifying one or more of the nucleotide sequences that correspond to the autoimmune disease;
producing antibodies or antigen-binding portions thereof encoded by one or more of the nucleotide sequences that correspond to the autoimmune disease; and
administering the one or more antibodies or antigen-binding portions thereof to said patient to inhibit or reduce symptoms of said autoimmune disease.

47. The method of claim 46, wherein the antibody-mediated autoimmune disease is multiple sclerosis, neuromyelitis optica, optic neuritis, transverse myelitis, acute disseminated encephalitis, systemic lupus erythematosus (SLE), rheumatoid arthritis, sjogren's disease, psoriasis, vasculitis, crohn's disease, or inflammatory bowel disease.

48. The method of claim 47, wherein the antibody-mediated autoimmune disease is characterized by type II or type III hypersensitivity.

49. The method of claim 47, wherein the antibody-mediated autoimmune disease is a demyelinating disease.

50. The method of claim 49, wherein the immunoglobulin-encoding genes or transcripts are VH4 genes or transcripts.

51. The method of claim 50, wherein the VH4 gene or transcript has at least two mutations with respect to the germline sequence at codons selected from 31B, 32, 40, 56, 57, 60, 81, and 89.

52. The method of claim 51, wherein the VH4 antibody binds to an antigen in human gray matter.

53. The method of claim 51 or 52, wherein the VH4 antibody has 3, 4, 5, or 6 mutations with respect to the germline sequence at codons selected from 31B, 32, 40, 56, 57, 60, 81, and 89.

54. The method of claim 53, wherein the VH4 antibody has mutations at two or more of codons 56, 57, and 81 with respect to the germline sequence, or at two or more of codons 40, 56, 81, and 89 with respect to the germline sequence.

55. The method of claim 53, wherein the VH4 antibody has mutations at one or more of codons 32, 40, 57, 60, and 89 with respect to the germline sequence.

56. The method of any one of claims 51 to 55, wherein the VH4 antibody has one or more of the following mutations:
codon 31B is R, N, D, P, K, G, A, or T, or is selected from R, N, and D;
codon 40 is S, L, or A;
codon 56 is selected from R, G, N, T, Y, H, D, and K, or is selected from N, T, and G;
codon 57 is A, I, D, S, or K, or is selected from A and I;
codon 81 is N, R, or M; and
codon 89 is selected from F, I, R, and L.

57. The method of claim 56, wherein the VH4 antibody has an R or N replacement at codon 81.

58. The method of claim 56, wherein the VH4 antibody has a T or R replacement at codon 56, optionally with the germline amino acid at codons 31B, 40, and 89, and which optionally binds astrocytes.

59. The method of any one of claims 50 to 58, wherein the VH4 germline is 4-04, 4-28, 4-30, 4-31, 4-34, 4-39, 4-59, 4-61 or 4-B.

60. The method of any one of claims 51 to 59, wherein the antibody has a set of mutations selected from Table 1.

61. The method of any one of claims 49 to 60, wherein the demyelinating disease is multiple sclerosis (MS).

62. The method of any one of claims 46 to 61, wherein the antibody produced is an antibody lacking an Fc.

63. The method of claim 62, wherein the antibody is a F(ab′)2 or Fab, a single chain antibody, or single chain variable fragment (scFv).

64. The method of any one of claims 46 to 63, wherein the antibody is formulated for administration by subcutaneous, intravenous, intramuscular, oral, or intrathecal administration.

65. A method for making an active agent for treating an antibody-mediated autoimmune disease, the process comprising:
determining nucleotide sequences in a patient sample for a plurality of immunoglobulin-encoding genes or transcripts;
identifying one or more of the nucleotide sequences that correspond to the autoimmune disease;
producing antibodies or antigen-binding portions thereof encoded by one or more of the nucleotide sequences that correspond to the autoimmune disease; and
formulating the one or more antibodies or antigen-binding portions thereof for delivery to said patient.

66. The method of claim 65, wherein the antibody-mediated autoimmune disease is multiple sclerosis, neuromyelitis optica, optic neuritis, transverse myelitis, acute disseminated encephalitis, systemic lupus erythematosus (SLE), rheumatoid arthritis, sjogren's disease, psoriasis, vasculitis, crohn's disease, or inflammatory bowel disease.

67. The method of claim 65, wherein the antibody-mediated autoimmune disease is characterized by type II or type III hypersensitivity.

68. The method of claim 66, wherein the antibody-mediated autoimmune disease is a demyelinating disease.

69. The method of claim 68, wherein the immunoglobulin-encoding genes or transcripts are VH4 genes or transcripts.

70. The method of claim 69, wherein the VH4 gene or transcript has at least two mutations with respect to the germline sequence at codons selected from 31B, 32, 40, 56, 57, 60, 81, and 89.

71. The method of claim 70, wherein the VH4 antibody binds to an antigen in human gray matter.

72. The method of claim 70 or 71, wherein the VH4 antibody has 3, 4, 5, or 6 mutations with respect to the germline sequence at codons selected from 31B, 32, 40, 56, 57, 60, 81, and 89.

73. The method of claim 72, wherein the VH4 antibody has mutations at two or more of codons 56, 57, and 81 with respect to the germline sequence, or at two or more of codons 40, 56, 81, and 89 with respect to the germline sequence.

74. The method of claim 72, wherein the VH4 antibody has mutations at one or more of codons 32, 40, 57, 60, and 89 with respect to the germline sequence.

75. The method of any one of claims 70 to 74, wherein the VH4 antibody has one or more of the following mutations:
codon 31B is R, N, D, P, K, G, A, or T, or is selected from R, N, and D;
codon 40 is S, L, or A;
codon 56 is selected from R, G, N, T, Y, H, D, and K, or is selected from N, T, and G;
codon 57 is A, I, D, S, or K, or is selected from A and I;
codon 81 is N, R, or M; and
codon 89 is selected from F, I, R, and L.

76. The method of claim 75, wherein the VH4 antibody has an R or N replacement at codon 81.

77. The method of claim 75, wherein the VH4 antibody has a T or R replacement at codon 56, optionally with the germline amino acid at codons 31B, 40, and 89, and which optionally binds astrocytes.

78. The method of any one of claims 69 to 77, wherein the VH4 germline is 4-04, 4-28, 4-30, 4-31, 4-34, 4-39, 4-59, 4-61 or 4-B.

79. The method of any one of claims 70 to 78, wherein the antibody has a set of mutations selected from Table 1.

80. The method of any one of claims 68 to 79, wherein the autoimmune disease is multiple sclerosis (MS).

81. The method of any one of claims 65 to 70, wherein the antibody produced is an antibody lacking an Fc.

82. The method of claim 81, wherein the antibody is a F(ab′)2 or Fab, a single chain antibody, or single chain variable fragment (scFv).

83. The method of any one of claims 65 to 82, wherein the oligonucleotide is formulated for administration by subcutaneous, intravenous, intramuscular, oral, or intrathecal administration.

84. A pharmaceutical formulation prepared by the method of any one of claims 65 to 83.

85. A method for producing a therapeutic antibody, comprising:
providing a first population of B cells from one or more subjects displaying an immune response against a malignancy or infectious disease, and a second population of B cells that do not display said immune response;
determining nucleotide sequences for a plurality of immunoglobulin-encoding genes or transcripts;
identifying one or more of the nucleotide sequences that correspond to the immune response; and
producing recombinant antibodies or antigen-binding portions thereof encoded by one or more of the nucleotide sequences that correspond to the immune response.

86. The method of claim 85, wherein the B cells displaying an immune response are from a patient having a malignancy.

87. The method of claim 86, wherein the malignancy is squamous or basal cell carcinoma, melanoma, biliary tract cancer, bladder cancer, bone cancer, brain or central nervous system cancer, breast cancer, cancer of the peritoneum, cervical cancer, colon or rectum cancer, cancer of the digestive system, endometrial cancer, esophageal cancer, eye cancer, cancer of the head and neck, gastric cancer, glioblastoma, neuroblastoma, liver cancer, kidney cancer, larynx cancer, leukemia, lung cancer, small-cell lung cancer, non-small cell lung cancer, adenocarcinoma of the lung, mesothelioma, myeloma, oral cavity cancer, ovarian cancer, pancreatic cancer, prostate cancer, retinoblastoma, rhabdomyosarcoma, salivary gland carcinoma, testicular cancer, thyroid cancer, uterine cancer, lymphoma, Hodgkin's lymphoma, non-Hodgkin's lymphoma, Cutaneous T-Cell Lymphoma, or B-cell lymphoma.

88. The method of claim 86 or 87, wherein the malignancy is a solid tumor.

89. The method of claim 88, wherein B cells are isolated from tumor tissue.

90. The method of any one of claims 85 to 89, wherein B cells are isolated from peripheral blood.

91. The method of any one of claims 85 to 90, wherein the first population of B cells are distinguished from the second population of B cells by their affinity for tumor cells or antigens.

92. The method of claim 91, wherein the second population of B cells are from the same patient(s) as the first population.

93. The method of claim 91, wherein the second population comprises B cells from patients that do not display an immune response against the tumor type.

94. The method of any one of claims 87 to 93, wherein the antibodies or antigen-binding portions thereof are recombinantly produced and administered to said patient(s) to target the tumor for destruction or as an imaging reagent.

95. The method of any one of claims 87 to 93, wherein the antibodies or antigen-binding portions thereof are recombinantly produced and administered to patients with the same tumor type for tumor destruction or as an imaging reagent.

96. The method of claim 85, wherein the subjects have an infectious disease.

97. The method of claim 96, wherein the infectious disease is a bacteria, virus, fungus, or parasite.

98. The method of claim 96 or 97, wherein the B cells are isolated from infected tissue.

99. The method of any one of claims 96 to 98, wherein B cells are isolated from peripheral blood.

100. The method of any one of claims 96 to 99, wherein the first population of B cells are distinguished from the second population of B cells by their affinity for pathogen cells or antigens.

101. The method of claim 100, wherein the second population of B cells are from the same patient(s) as the first population.

102. The method of claim 100, wherein the second population comprises B cells from patients that do not display a productive immune response against the pathogen.

103. The method of any one of claims 96 to 102, wherein the antibodies or antigen-binding portions thereof are produced and administered to one or more patients to target the pathogen.

104. The method of any one of claims 85 to 103, wherein nucleotide sequences are evaluated based on heavy or light chain gene families, V-D-J usage, and mutations within the heavy and/or light chain as compared to the germline sequence.
